(19) United States
(12) Patent Application Publication
(10) Pub. No.: US 2019/0015104 A1
(43) Pub. Date: Jan. 17, 2019

(54) CONNECTOR AND METHOD FOR COUPLING ANATOMICAL WALLS

(71) Applicant: NUHEART AS, Bergen (NO)

(72) Inventors: Vegard TUSETH, Bergen (NO); Matthew KEILLOR, Inver Grove Heights, MN (US); Philip HAARSTAD, St Louis Park, MN (US); Shawn PATTERSON, Bergen (NO)

(21) Appl. No.: 16/068,624

(22) PCT Filed: Jan. 6, 2017
(86) PCT No.: PCT/EP2017/050275

§ 371 Date: Jul. 6, 2018

(30) Foreign Application Priority Data
Jan. 8, 2016 (US) 14991662
Jan. 8, 2016 (US) 14991675

(51) Int. Cl. A61B 17/11; A61M 1/10; A61M 1/12

(52) U.S. Cl. CPC A61B 17/11; A61M 1/101; A61B 2017/1139; A61B 2017/1107; A61M 1/122

(57) ABSTRACT

A connector is provided for fluid communication between two anatomical compartments through at least one anatomical wall, wherein the connector includes a neck adapted and configured to be positioned across the anatomical wall(s); a primary element for securing the neck across the anatomical wall(s); and a secondary element for securing the neck across the anatomical wall(s). A method is also provided for coupling two anatomical walls using said connector.

### FIELD OF THE INVENTION

The present invention generally relates to the field of intracorporeal medical devices. More specifically to a connector for assisting fluid communication between two anatomical compartments and for supporting, maintaining and protecting the anatomical walls of the compartments in close contact with each other. The present invention is particularly useful in the context of minimally invasive transcatheter and/or percutaneous procedures, such as those described in PCT application No. PCT/EP2015/055578, entitled “PERCUTANEOUS SYSTEM, DEVICES AND METHODS” filed 17 Mar. 2015 and expressly incorporated herein by reference in its entirety.

### BACKGROUND

In PCT/EP2015/055578, the Inventor describes an intracorporeal connector for fluid communication between a first and a second anatomical compartment, which is configured to anchor a fluid regulation device such as a pump, through one or more anatomical walls. The connector comprises an anchor adapted and configured to lie against the anatomical wall of the second compartment, a neck adapted to be positioned through the anatomical wall(s) and a shield adapted and configured to expand in the second compartment. The connector can be inserted through a delivery catheter in a folded state and deployed at the implantation site. The deployed connector pulls the anatomical walls in contact with one another, prevents the leaking of blood into the interstitial space between the anatomical walls and allows the pump to be anchored to the anatomical walls.

It has however been observed that there is a potential risk of blood leak during the implantation process itself. As the folded connector exits the delivery catheter, the anchor unfolds until it reaches its working position against the anatomical wall. Similarly, the shield unfolds until it reaches its working position. There is therefore a time period during which the anatomical walls are punctured, and the puncture is supported only by the delivery catheter until the anchor and shield are fully deployed. Any slight unintentional movement can potentially lead to blood leak into the interstitial space between the anatomical walls. In extreme cases, in particular in the case of accidental movements, the delivery catheter could become dislodged and the puncture exposed, resulting in severe blood loss. On the other hand, accelerating the deployment could equally lead to injury to the patient, in particular if the anchor and/or shield were designed to accelerate the deployment, they could snap into a deployed configuration against the anatomical walls and result to injury. Also, the anchor and/or shield could be deployed before the practitioner has had the time to correctly position them.

Consequently, a compromise must be found between a shorter deployment time so as to minimise the time during which the anatomical walls are left unsupported, and allowing enough time to the medical practitioner to correctly implant the connector.

There is therefore the need for a new device with improved accuracy and safety, a device which is more forgiving in that it allows more time for the medical practitioner to correctly position the connector, whilst minimising the risk of blood leak.

### SUMMARY OF THE INVENTION

It is an object of this invention to mitigate problems such as those described above.

According to a first aspect of the invention, there is provided a connector for fluid communication between two anatomical compartments through at least one anatomical wall, wherein the connector comprises a neck adapted and configured to be positioned across the anatomical wall(s); primary means for securing the neck across the anatomical wall(s); and secondary means for securing the neck across the anatomical wall(s).

Thus, the invention seeks to provide a connector for fluid communication between two anatomical compartments and which can be implanted in a safe and controlled manner. The secondary securing means is provided as a safety feature to prevent fluid loss during the delivery and implantation process of the connector. As such, it provides a connector, the manipulation of which is more forgiving. Once the neck is positioned across the anatomical wall(s), the connector is secured to the anatomical wall(s) by the primary securing means so that it does not become dislodged from the wall due to blood flow, patient's movements, manipulations from the medical practitioner and the like. The primary securing means also performs the function of supporting the anatomical wall(s). This is particularly important when manipulating, inserting and/or implanting of further medical devices through the anatomical wall(s) as such manipulation, insertion and/or manipulation could lead to injury or trauma of the anatomical wall(s), which in turn could result in potentially fatal blood loss. It is also important to note that blood pressure in certain parts of the human body (e.g. the heart) can be significant and the connector supports and protects the anatomical wall(s) against such pressure.

The connector according to the present invention supports, maintains and protects the anatomical wall(s). The connector minimises the risk of damage, rupture, trauma, injury and perforation of the anatomical wall(s).

The anatomical compartments may be separated by one wall, for example in the case of two adjoining compartments and the connector prevents any uncontrolled fluid flow between the two compartments. The present invention is particularly advantageous when the anatomical compartments are separated by two anatomical walls, i.e. in the case of two adjacent but remote compartments normally separated by a gap. In this embodiment, the connector prevents any uncontrolled blood loss into the compartments and said gap.

The connector may be used to establish fluid communication between two anatomical compartments, more preferably controlled blood flow between compartments of the cardio-vascular system, although other fluids and anatomical compartments are envisaged. Compartments of the cardio-vascular system include for example the left atrium, the right atrium, the left ventricle, the right ventricle, the aorta, the pulmonary artery, the vena cava as well as arteries, veins and other compartments of the peripheral vascular system. More advantageously, the connector of the present invention connects two adjacent but remote compartments so that in its working configuration, the connector is positioned across two anatomical walls.

Preferably, the connector can be arranged into a delivery configuration and into a working configuration in which the neck is secured across the anatomical wall(s). For example, the connector may be collapsible and/or extendable so that, in a first configuration, it facilitates delivery to the implantation site and across the anatomical wall(s) and, in a second configuration, it connects the two compartments for fluid communication. This enables the connector to be used in smaller patients (children) and/or patients with compromised and/or narrower delivery paths.

Preferably, the primary securing means and/or the secondary securing means are movable from a delivery configuration to a working configuration in which the neck is secured across the anatomical wall(s). The primary securing means may have one or more elements which are movable relative to the neck and/or the secondary securing means may have one or more elements which are movable relative to the neck.

Preferably, the secondary securing means is capable of securing the neck across the anatomical wall(s) before the primary securing means secures the neck across the anatomical wall(s). Thus, the secondary securing means can act as an interim securing means, before the primary securing means becomes effective and as an additional safety against potential fluid and/or blood leaks once the primary securing means is in its working configuration.

Preferably, the primary securing means and/or the secondary securing means comprise a plurality of arms. The use of arms as securing means allows for more accurate control of the implantation process in terms of predictability, homogeneity and diffusion of the pressure across the structure, controlled delivery speed and safety control.

The arms of the primary securing means and/or of the secondary securing means may be deployable from a delivery configuration to a working configuration. Similarly, the arms may be foldable and/or compressable from a working configuration to a delivery configuration. Within the context of the invention, a delivery configuration means a configuration which is suitable for the connector to be delivered to the implantation site and which differs from a working configuration, in which the connector connects the two anatomical compartments for fluid communication therebetween and is secured to the anatomical wall(s). The mechanism may be any mechanism which enables the connector to switch from one configuration to the other, and include deployment, compression, folding, extending, retracting and the like.

Preferably, the arms extend from one or both ends of the neck. For example, the primary securing means may comprise a set of arms extending from one end of the neck; or a first set of arms extending from a first end of the neck and a second set of arms extending from a second end of the neck. The secondary securing means may comprise a set of arms extending from one end of the neck; or a first set of arms extending from a first end of the neck and a second set of arms extending from a second end of the neck. Preferably, the primary securing means comprises two sets of arms, each set extending from an end of the neck. Preferably, the secondary securing means comprises two sets of arms, each set extending from an end of the neck. These features ensure that the connector is suitably secured to the anatomical wall(s) and that the neck is suitably buried across the anatomical wall(s) as a safety against potential blood losses and so as to prevent any potential injury caused by the neck extending into the compartment(s).

In a preferred embodiment, the primary and/or secondary securing means are integrally formed with the neck. For example, the arms may be integrally formed with neck. This firstly ensures a smooth transition between the arms and the neck, with no additional hinging mechanism which may potentially cause injury during the deployment process. In addition, this structure is less complex to manufacture. The connector may for example comprise or consist of a shape memory material, such that the arms can be pushed inwards (relative to the neck) into a delivery configuration and extended outwards into a working configuration when it exits e.g. a delivery catheter. This allows for a gentle, continuous and smooth securing process and bringing together of anatomical walls with limited risk of injury.

Preferably, each deployable arm is independently movable from the other arms. More generally, the primary securing means may be independently movable from the secondary securing means. If the primary and/or secondary securing means comprises one or more sets of arms, then each set of arms may be independently movable from other sets of arms. For example, one or more arms could be non-movable whilst one or more arms are movable. In a preferred embodiment, the speed of deployment of a securing means may be faster than the speed of deployment of another securing means.

In a preferred embodiment, the arms can extend substantially parallel to the longitudinal axis of the neck or at an angle sufficient to fit into a catheter in a delivery configuration. Preferably, in the working configuration, the arms extend substantially perpendicular to the longitudinal axis of the neck or at an angle sufficient to partially or completely contact the anatomical wall.

Preferably, the arms of the secondary securing means are adapted and configured to fit, in their working configuration, in a delivery catheter or other transcatheter or percutaneous delivery device. In a preferred embodiment, the arms are partially deployed or completely deployed in their working configuration whilst in the delivery device. For example, the arms may be of dimensions such that they fit in the delivery device in a partially or completely deployed configuration. The arms in their full working configuration may have dimensions smaller than the inner dimensions of the delivery device so that they fit in full working configuration in the delivery device. Alternatively, the arms in their full working configuration may have dimensions greater than the inner dimensions of the delivery device so that they fit is a partly deployed configuration. Thus, the secondary securing means can exit the delivery device and be implanted in a partly deployed or fully deployed configuration so that they can promptly or immediately secure the connector to the anatomical walls. This is particularly advantageous when the primary securing means requires more time to deploy into its working configuration and the primary securing means acts as a safety securing means until the primary securing means is fully effective.

Preferably, each arm forms a loop. It is not required that all arms form a loop, and for example, some of the arms may form a loop. This is particularly advantageous to provide further support to the anatomical wall, i.e. by increasing the contacting area without compromising the efficiency and versatility of the connector. It is also useful to homogenise and/or to spread the pressure exerted by the arms onto the anatomical wall.

Within the context of the invention, a loop means that the beginning of the arm and adjacent the end of the arm. For example, an arm can form a loop by having both ends connected to the neck of the connector. The loop can be partly or fully circular or oval. Rounded loops are preferred to angular loops to minimise the risk of injury to the patient. The loop can for example comprise two or more substantially linear portions, which may or may not be parallel to each other. It is also envisaged an embodiment wherein a securing means consists of a single arm which forms a loop or a shape suitable to secure the neck to the anatomical wall(s). Therefore, the present connector is versatile in that its shape can be adjusted for different requirements including requirements resulting from uses, implantation sites, patient anatomy and the like.

Preferably, each arm comprises one or more eyelets to provide an improved grip to the anatomical wall(s) and to prevent slipping of the arms on the surface of the anatomical wall(s). This, in turn, minimises the risk of the connector becoming displaced or dislodged and the risk of trauma to the anatomical tissues and increases safety against blood leaks. The eyelets also provide a mechanism for securing the connector to the delivery device for secure and accurate delivery.

The arms may be substantially flat to provide increased support area. They may be rounded or tubular to minimise the risk of trauma and injury to the patient's tissues and anatomical walls.

Preferably, a web of material or mesh extends between the arms. This feature may be advantageous in increasing the contacting area of the connector to provide further support and/or to spread the pressure exerted by the arms. The material may also act as a shield to prevent tissues and/or walls from being pulled with the fluid flow.

In another embodiment, the primary securing means and/or the secondary securing means may be devoid of arms. For example, it may comprise a shield or and anchor as illustrated in PCT application No. PCT/EP2015/055578, incorporated herein by reference. It may comprise a web, mesh and/or other material. Preferably, the material is flexible or semi-rigid. Preferably, the primary securing means and/or the secondary securing means are adapted and configured to be deployable from a delivery configuration to a working configuration. It may be made of a shape memory material. The primary securing means and/or the secondary securing means may be substantially flat or substantially bowl- or umbrella-shaped. The primary securing means and/or the secondary securing means may also act as a shield to prevent tissues and other elements hindering fluid passage through the neck.

The securing means and the neck may be made of the same or different material, and may have the same or different thicknesses. It may also be envisaged that the neck may be made of different materials and/or materials of different thicknesses so that the thickness of the neck would vary for different sections.

In another embodiment of the present invention, the primary securing means and/or the secondary securing means form a diffusor to improve and/or enhance fluid flow from one compartment to the other. Preferably, the securing means form a cavity or compartment at one or both ends of the neck. More preferably, the securing means form a cavity or compartment in the receiving compartment so as to direct and/or improve the flow pattern of the blood released from the pump into the receiving compartment. Preferably, the securing means comprise of consists of arms, mesh and/or grid, which is optionally coated so as to form a cavity or compartment.

Preferably, the connector is configured and adapted to be coupled to an intracorporeal pump. Preferred pumps include but are not limited to the fluid regulating device described in PCT application No. PCT/EP2015/055578, incorporated herein by reference. The connector is particularly advantageous for coupling a pump intracorporeally, i.e. not pre-coupled with a pump outside the patient's body. However, it is envisaged in another embodiment that the connector may be integrally formed with a medical device, such as an intracorporeal pump.

Preferably, the neck comprises or is made of a resilient, flexible or semi-rigid material. Alternatively, the neck comprises or is made of a resilient, flexible or semi-rigid structure, such as a mesh structure. The neck is preferably able to expand to receive or remove a pump and to compress back to retain and secure the pump to the connector. It should preferably be rigid enough to maintain the aperture in the anatomical wall(s) opened. The neck is preferably able to expand radially to receive, secure and release the pump and other devices as required; and/or longitudinally according to the thickness of the anatomical walls and the size of the gap between the anatomical walls to prevent too much pressure being exerted onto the anatomical wall and therefore injury to the patient. In a preferred embodiment, the neck comprises or consists of a substantially diamond-patterned mesh or web.

The length of the neck can be adjusted to substantially match the thickness of the anatomical wall(s) and any gap therebetween; or the neck can be longitudinally expandable to accommodate the thickness of the anatomical wall(s) and any gap therebetween. However, for optimum protection against blood or fluid leaks, the neck is covered or coated with an impermeable membrane, which is preferably biocompatible. Alternatively or additionally, the neck is partially or completely surrounded by a ring of impermeable material, such as rubber.

Suitable materials include, but are not limited to, polymers such as polytetrafluoroethylene (pTFE), silicon, polyvinylidene fluorinated (PVDF) polymers, polyurethane and combinations thereof. Suitable application techniques include, but are not limited to, electro-spinning, electro-spun, dip coating techniques.

Preferably, the neck comprises a tapered portion. This feature allows the pump or other medical device to be easily inserted into and across the neck, without the application of force, and to retain and secure the device across the neck of the connector. In particular, this feature improves safety in that the taper prevents the wall(s) from sliding back over the neck, thereby releasing the anatomical walls from contact, reopening the space therebetween and risking potentially fatal blood leakage. In an alternative embodiment, the neck is substantially hourglass-shaped, i.e. the neck comprises a cross section of smaller diameter between a distal and a proximal section of larger diameter. Thus, the anatomical walls are compressed at the waist of the hour-glass shaped neck and the effective size of the neck to push the pump through is minimised. In another embodiment, the connector is devoid of a channel. It may for example comprise or consist of a ring-shaped element.

According to a second aspect of the invention, there is provided a connector for fluid communication between two anatomical compartments through at least one anatomical wall, wherein the connector comprises primary means for securing the connector across the anatomical wall(s); and secondary means for securing the connector across the anatomical wall(s). In other words, there is provided a connector as described in any one of the preceding paragraphs, without a neck.

Preferably, the connector comprises means for connecting the primary securing means to the secondary securing means. For example, the connector may comprise at least one ring connected to the primary securing means and the secondary securing means. The ring is preferably circular, but may be any other suitable shape.

The advantage of a connector with no neck portion is that the anatomical walls are pressed against each other and maintained in contact by the securing means, so that blood flows directly from one compartment to the other without passing through a neck channel and without the risk of leaking into the gap between the two anatomical wall(s). In this embodiment, the primary and secondary securing means may have any of the features and characteristics of the primary and secondary securing means as described in the preceding paragraphs with respect to a connector with a neck.

According to a third aspect of the invention, there is provided a method for coupling two anatomical walls using a connector as described in any one of the preceding paragraphs, with or without a neck. The method may also be a method for implanting a connector through one or more anatomical walls.

A method for coupling two anatomical walls using a connector comprising primary means for securing the connector across the anatomical wall(s); and secondary means for securing the connector across the anatomical wall(s), preferably comprises the step of securing the connector to the anatomical wall(s) using the secondary securing means prior to securing the connector to the anatomical wall(s) using the primary securing means.

The present method enables the medical practitioner to safely position the connector across the anatomical wall(s) with minimum risk of blood loss. During the insertion process, the secondary securing means initially secures the connector to the anatomical wall(s) so the medical practitioner is able to manipulate the connector into the correct position before the primary securing means finally secures the connector to the wall(s).

Preferably, the connector can be arranged into a delivery configuration and into a working configuration in which the neck is secured across the anatomical wall(s).

Preferably, the primary means and/or the secondary means are movable from a delivery configuration to a working configuration in which the neck is secured across the anatomical wall(s).

Preferably, the method comprises the step of delivering the connector across the anatomical wall(s) using a delivery catheter or device. The connector may therefore be inserted in the delivery device in its delivery configuration and deploy into its working configuration as it exits the delivery device.

Preferably, the primary securing means comprises a first set of arms and a second set of arms; and the secondary securing means comprises a first set of arms and a second set of arms.

Preferably, the method comprises the step of deploying the first set of arms of the secondary securing means in the distal compartment. In other words, sequentially, the first set of arms of the secondary securing means is deployed into a working configuration before any other arms. Within the context of the invention, the terms “distal” and “proximal” are relative to the direction of insertion, e.g. from a proximal compartment to a distal compartment.

Preferably, the first set of arms of the secondary securing means is partially or completely pre-deployed in the delivery catheter.

Preferably, the first set of arms of the secondary securing means have smaller dimensions than the first set of arms of the primary securing means. This can result in the first set of arms of the secondary securing means being partially or completely deployed in the delivery device so that it can be effective as it soon as possible as it exits the delivery device; and/or, more generally, in the first set of arms of the secondary securing means being in its working configuration before the first set of arms of the primary securing means reaches its working configuration.

Preferably, the method comprises the step of deploying the first set of arms of the primary securing means in the distal compartment.

Preferably, the method comprises the step of deploying the second set of arms of the second securing means in the proximal compartment.

Preferably, the second set of arms of the secondary securing means is partially or completely pre-deployed in the delivery catheter.

Preferably, the second set of arms of the secondary securing means have smaller dimensions than the second set of arms of the primary securing means. This can result in the second set of arms of the secondary securing means being partially or completely deployed in the delivery device so that it can be effective as it soon as possible as it exits the delivery device; and/or, more generally, in the second set of arms of the secondary securing means being in its working configuration before the second set of arms of the primary securing means reaches its working configuration.

Preferably, the method comprises the step of deploying the second set of arms of the primary securing means in the proximal compartment.

Preferably, the first set of arms of the primary securing means have smaller dimensions that the second set of arms of the primary securing means. This feature provides optimum support to the anatomical wall(s) against the pressure exerted by the blood flow.

In an embodiment of the invention, the connector is coupled to the insertion device so as to improve safety. In this embodiment, no catheter or wire exchange is requires so that the implantation can be effected promptly.

Thus, the device according to the present invention enables the medical practitioner to safely implant and position the connector through the anatomical wall(s).

### LIST OF EMBODIMENTS

The following is a non-limiting list of potential embodiments of the present invention, set forth as embodiment groups (each an “Embodiment”). Additional embodiments of the invention are possible, as set forth throughout this specification and the drawings.

### Embodiment 1

A connector for fluid communication between two anatomical compartments through at least one anatomical wall, wherein the connector comprises a neck adapted and configured to be positioned across the anatomical wall(s); primary means for securing the neck across the anatomical wall(s); and secondary means for securing the neck across the anatomical wall(s).

### Embodiment 2

The connector according to Embodiment 1, wherein the connector can be arranged into a delivery configuration and into a working configuration in which the neck is secured across the anatomical wall(s).

### Embodiment 3

The connector according to Embodiment 1 or 2, wherein the primary securing means and/or the securing secondary means are movable from a delivery position to a working position in which the neck is secured across the anatomical wall(s).

### Embodiment 4

The connector according to any preceding Embodiment, wherein the secondary securing means is capable of securing the neck across the anatomical wall(s) before the primary securing means secures the neck across the anatomical wall(s).

### Embodiment 5

The connector according to any preceding Embodiment, wherein the primary securing means and/or the secondary securing means comprise a plurality of arms.

### Embodiment 6

The connector according to Embodiment 5, wherein the arms of the primary securing means and/or of the secondary securing means are deployable from a delivery configuration to a working configuration.

### Embodiment 7

The connector according to Embodiment 5 or 6, wherein each deployable arm is independently deployable.

### Embodiment 8

The connector according to any one of Embodiments 5 to 7, wherein the arms extend from one or both ends of the neck.

### Embodiment 9

The connector according to any one of Embodiments 5 to 8, wherein the primary securing means comprises two sets of arms, each set extending from an end of the neck.

### Embodiment 10

The connector according to any one of Embodiments 5 to 9, wherein the secondary securing means comprises two sets of arms, each set extending from an end of the neck.

### Embodiment 11

The connector according to any one of Embodiments 5 to 10, wherein the arms of the secondary securing means are adapted and configured to fit, in their working configuration, in a delivery catheter.

### Embodiment 12

The connector according to any one of Embodiments 5 to 11, wherein a web of material extends between the arms.

### Embodiment 13

The connector according to any one of Embodiments 5 to 12, wherein each arm forms a loop.

### Embodiment 14

The connector according to any one of Embodiments 5 to 13, wherein each arm comprises one or more eyelets.

### Embodiment 15

The connector according to any preceding Embodiment wherein the connector is configured and adapted to be coupled to an intracorporeal pump.

### Embodiment 16

The connector according to Embodiment 15, wherein the neck is flexible.

### Embodiment 17

The connector according to Embodiment 15 or 16, wherein the neck comprises is formed of a mesh structure.

### Embodiment 18

The connector according to any one or Embodiments 15 to 17, wherein the neck comprises a tapered portion.

### Embodiment 19

The connector according to any one or Embodiments 15 to 18, wherein the neck is covered or coated with a biocompatible impermeable membrane.

### Embodiment 20

The connector according to any preceding Embodiment, wherein the connector enables fluid communication between at least two anatomical walls.

### Embodiment 21

The connector according to any preceding Embodiment, wherein the primary securing means form a diffusor.

### Embodiment 22

A connector for fluid communication between two anatomical compartments through at least one anatomical wall, wherein the connector comprises primary means for securing the connector across the anatomical wall(s); and secondary means for securing the connector across the anatomical wall(s).

### Embodiment 23

The connector according to Embodiment 22, further comprising at least one ring connected to the primary securing means and the secondary securing means.

### Embodiment 24

An intracorporeal pump integrally formed with a connector according to any preceding Embodiment.

### Embodiment 25

A method for coupling two anatomical walls using a connector comprising primary means for securing the connector across the anatomical wall(s); and secondary means for securing the connector across the anatomical wall(s), the method comprising the step of securing the connector to the anatomical wall(s) using the secondary securing means prior to securing the connector to the anatomical wall(s) using the primary securing means.

### Embodiment 26

The method according to Embodiment 25, wherein the connector can be arranged into a delivery configuration and into a working configuration in which the neck is secured across the anatomical wall(s).

### Embodiment 27

The method according to Embodiment 25 or 26, wherein the primary means and/or the secondary means are movable from a delivery position to a working position in which the neck is secured across the anatomical wall(s).

### Embodiment 28

The method according to any one of Embodiments 25 to 26, wherein comprising the step of delivering the connector across the anatomical wall(s) using a delivery catheter.

### Embodiment 29

The method according to any one of Embodiments 25 to 27, wherein the primary securing means comprises a first set of arms and a second set of arms; and the secondary securing means comprises a first set of arms and a second set of arms.

### Embodiment 30

The method according to Embodiment 29, comprising the step of deploying the first set of arms of the secondary securing means in the distal compartment.

### Embodiment 31

The method according to any one of Embodiments 29 to 30, wherein the first set of arms of the secondary securing means is pre-deployed in the delivery catheter.

### Embodiment 32

The method according to any one of Embodiments 29 to 31, wherein the first set of arms of the secondary securing means have smaller dimensions than the first set of arms of the primary securing means.

### Embodiment 33

The method according to any one of Embodiments 29 to 32, comprising the step of deploying the first set of arms of the primary securing means in the distal compartment.

### Embodiment 34

The method according to any one of Embodiments 29 to 33, comprising the step of deploying the second set of arms of the second securing means in the proximal compartment.

### Embodiment 35

The method according to one of Embodiments 29 to 34, wherein the second set of arms of the secondary securing means is pre-deployed in the delivery catheter.

### Embodiment 36

The method according to one of Embodiments 29 to 35, wherein the first set of arms of the secondary securing means have smaller dimensions than the second set of arms of the primary securing means.

### Embodiment 37

The method according to any one of Embodiments 29 to 36, comprising the step of deploying the second set of arms of the primary securing means in the proximal compartment.

### Embodiment 38

The method according to any one of Embodiments 29 to 37, wherein the second set of arms of the primary securing means have smaller dimensions that the second set of arms of the primary securing means.

In the above list of embodiments of the invention, each listed Embodiment, as a group of embodiments, comprises a single specific embodiment and/or plural specific embodiments, as specified in the particular combination of embodiments for each Embodiment group.

### BRIEF DESCRIPTION OF THE DRAWINGS

The invention will be further described with reference to the drawings and figures, in which

FIG. 1 is a schematic representation (side view) of a first connector according to the present invention in its working configuration;

FIG. 2 is a schematic representation (top view) of the connector as shown in FIG. 1;

FIGS. 3A and 3B are partial schematic representations of the connector as shown in FIG. 1;

FIG. 4 is a schematic representation of the connector as shown in FIG. 1 in its delivery configuration;

FIG. 5 is a schematic representation (perspective view) of the connector as shown in FIG. 1—cut and laid flat;

FIG. 6 are schematic representations (side views) of securing arms for use in connectors according to the present invention;

FIGS. 7A and 7B are schematic representations (top views) of securing arms for use in connectors according to the present invention;

FIG. 8 is a schematic representation of a second connector according to the present invention—cut and laid flat;

FIGS. 9A and 9B are schematic representations (perspective views) of a third connector according to the present invention, coupled with a pump;

FIG. 9C is a schematic representation (side view) of the connector as shown in FIG. 9A;

FIG. 10 is a schematic representation (bottom view) of the connector as shown in FIG. 9A;

FIG. 11 is a partial schematic representation of the connector as shown in FIG. 9A;

FIG. 12 is a schematic representation (side view) of a fourth connector according to the present invention in its working configuration;

FIG. 13 is a schematic representation (side view) of a fifth connector according to the present invention in its working configuration; and

FIGS. 14A to 14F illustrate a method for securing a connector according to the present invention across two anatomical walls.

### DETAILED DESCRIPTION

The invention is described by way of examples, which are provided for illustrative purposes only. These examples should not be construed as intending to limit the scope of protection that is defined in the claims. For example, although various aspects have been described with respect to the heart and the circulatory system, this is not intended to be limiting, and is merely performed to provide an example of implementation. Aspects disclosed herein may be utilised in any medical device implantable within the human body, for example in the cardiovascular system, respiratory system, gastric system, neurological system, and the like, some examples including implantable pumps and drug delivery pumps. As used herein, the term “means” can be equivalently expressed as, or substituted with, any of the following terms: device, apparatus, structure, part, sub-part, assembly, sub-assembly, machine, mechanism, article, medium, material, appliance, equipment, system, body or similar wording.

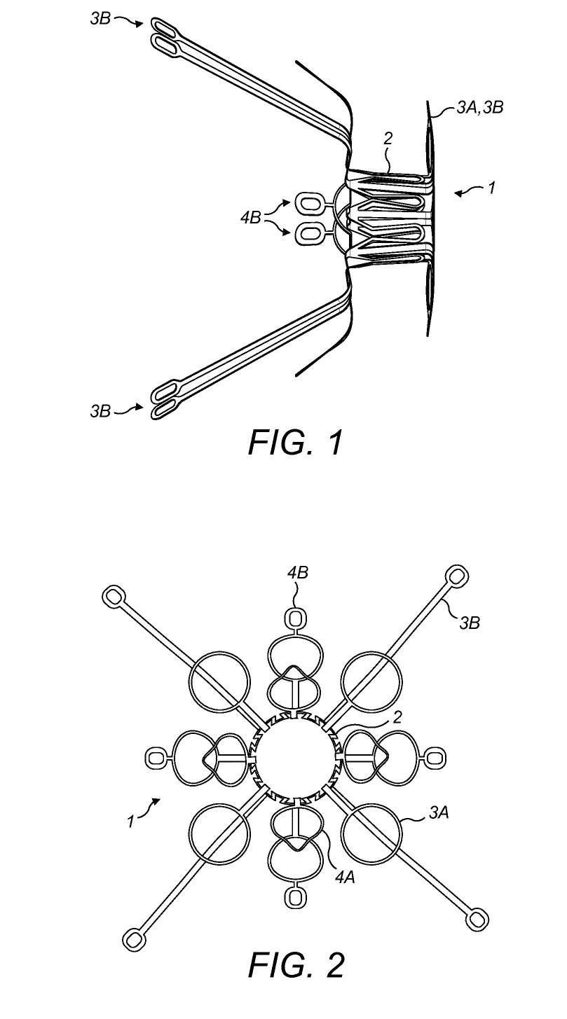

Referring to FIG. 1, there is illustrated a connector 1 for fluid communication between two anatomical compartments C through at least one anatomical wall W, wherein the connector 1 comprises a neck 2 adapted and configured to be positioned across the anatomical wall(s) W; primary securing means 3A, 3B for securing the neck 2 across the anatomical wall(s) W; and secondary securing means 4A, 4B for securing the neck 2 across the anatomical wall(s) W.

In this embodiment, the two compartments C are the left atrium C₁ and the ascending aorta C₂, and the connector implantation site is at a location where the left atrium C₁ and the aorta C₂ are separated by the wall W₁ of the left atrium C₁ and the aortic wall W₂ (i.e. two anatomical walls). The exemplified direction of insertion is from the left atrium C₁ (proximal compartment) to the aorta C₂ (distal compartment).

The primary and secondary securing means each comprises a plurality of arms 3,4 extending from the neck 2 of the connector 1. The primary securing means comprises a first set of arms 3A extending from one end of the neck (the aortic side) and a second set of arms 3B extending from the other end of the neck (the atrial side). The secondary securing means comprises a first set of arms 4A extending from one end of the neck (the aortic side) and a second set of arms 4B extending from the other end of the neck (the atrial side).

Hereinafter, the arms of the first set of arms 3A of the primary securing means will be referred to as long aortic arms 3A; the arms of second set of arms 3B of the primary securing means will be referred to as long atrial arms 3B; the arms of the first set of arms 4A of the secondary securing means will be referred to as short aortic arms 4A; the arms of second set of arms 4B of the secondary securing means will be referred to as short atrial arms 4B; bearing in mind that the present invention is not limited to left atrium/aorta procedures.

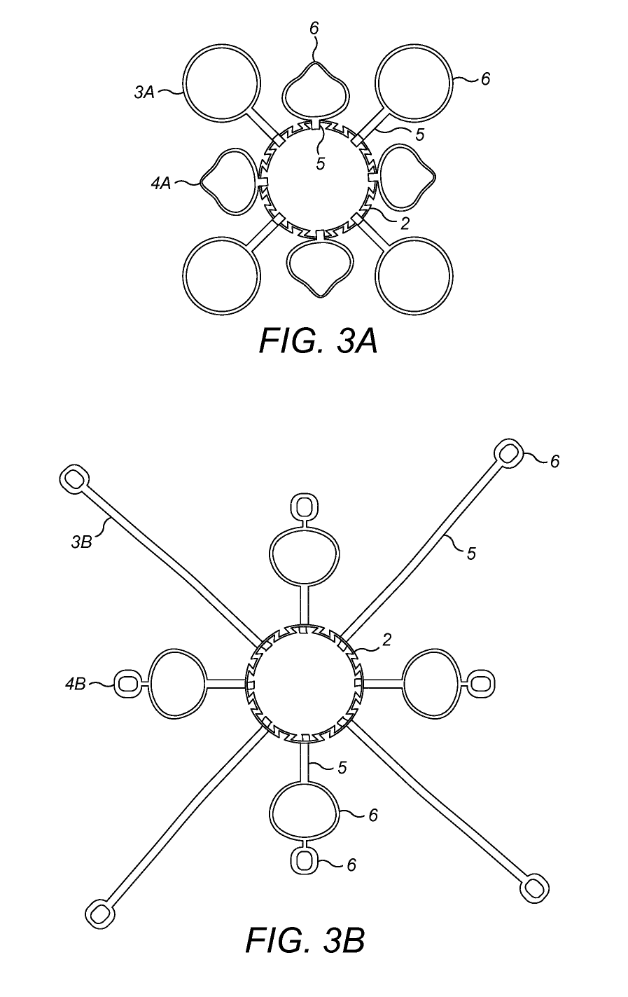

With reference to FIGS. 3A and 3B, the long aortic arms 3A comprise a substantially linear portion 5 connecting an eyelet 6 to the neck 2. The long atrial arms 3B are longer than the long aortic arms 3A, and comprise a substantially linear portion 5 connecting an eyelet 6 to the neck 2. The short aortic arms 4A comprise a substantially linear portion 5 connecting an eyelet 6 to the neck 2. The short atrial arms 4B are longer than short aortic arms 4A, and comprise a substantially linear portion 5 connecting two eyelets 6 to the neck 2.

FIG. 1 shows a connector 1 according to the present invention in its working configuration. The long and short aortic arms 3A, 4A extend substantially perpendicularly from the aortic end of the neck 2 so as to lie against and support the aortic wall W₂. Part of the linear portion 5 of the long and short atrial arms 3B, 4B extend substantially perpendicularly from the atrial end of the neck 2 so as to lie against and support the atrial wall W₁, whilst the remaining portion of the long and short atrial arms 3B, 4B extend away from the atrial wall W₁ so as to form a shield which prevents surrounding tissues from hindering the channel of the neck 2.

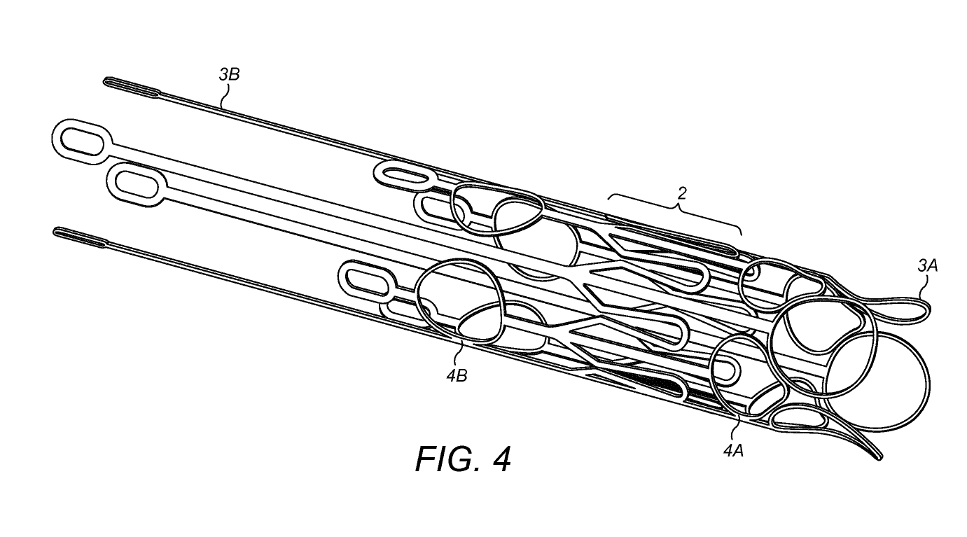

FIG. 4 shows a connector 1 according to the present invention in its delivery configuration, in which the arms 3A, 3B, 4A, 4B extend substantially parallel relative to the neck 2 so as to fit, for example, in a delivery catheter 7.

The arms 3A, 3B, 4A, 4B are made of or comprise a shape memory metal, or other material which allows the arms to adopt a delivery configuration and a working configuration (which preferably differ from each other). The arms may or may not be made of the same material as the neck 2, preferably the same material if the arms and neck are integrally formed. In the examples included herein, the arms of the primary and secondary securing means are integrally formed with the neck 2 of the connector 1 but it could be envisaged that one or more or all arms are formed separately and movably connected to the neck 2.

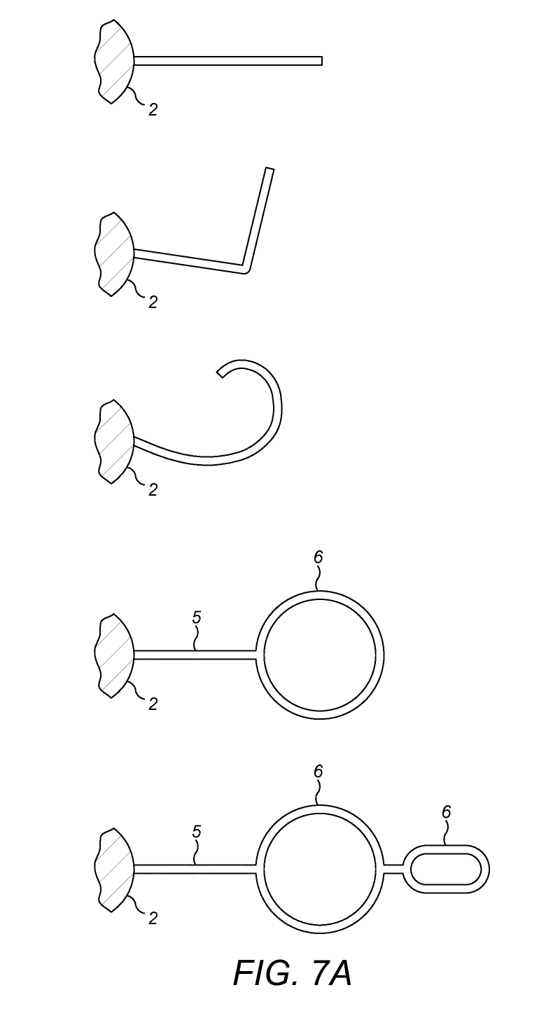

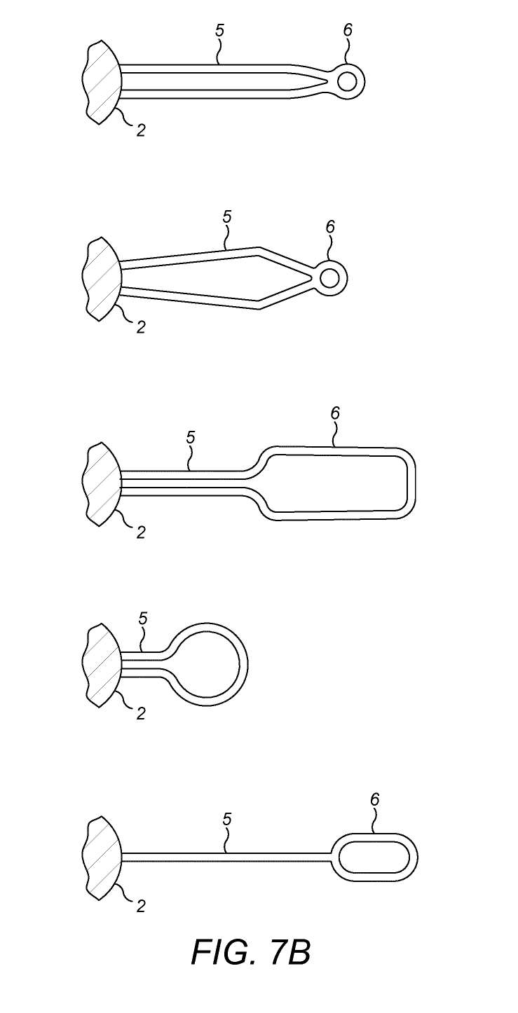

Examples of arm shapes are illustrated in FIGS. 6, 7A and 7B. The shape and dimensions of the arms can be adjusted depending on the requirements. The arms may be partially or completely straight, curved or bent relative to the anatomical wall W (see FIG. 6). They may comprise one or more linear portions 6 and/or one or more eyelets 5 (see FIGS. 7A and 7B). When the arms comprise two or more linear portions 6 (preferably two), the linear portions may be substantially parallel to each other.

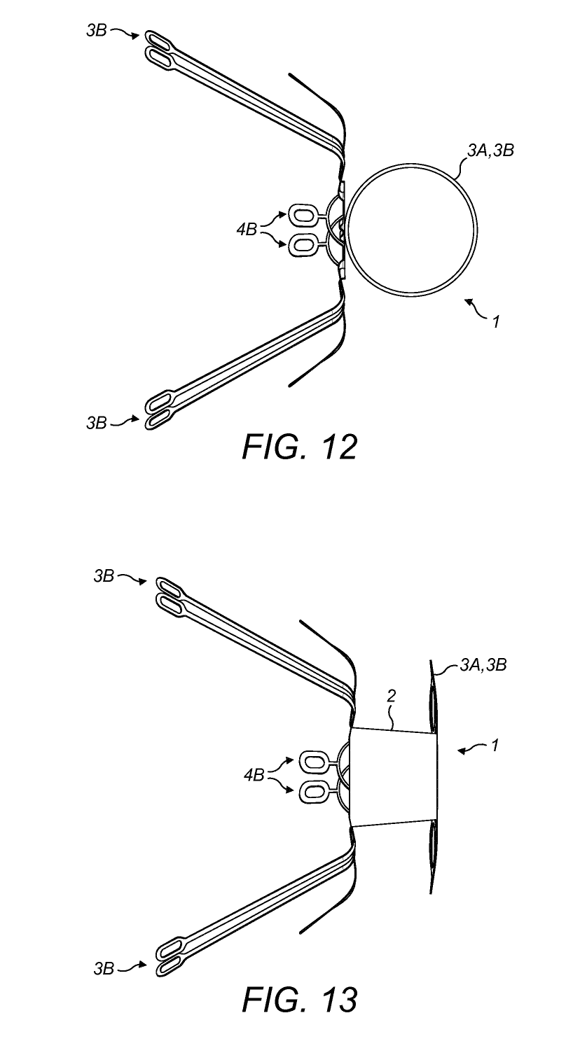

In this embodiment, the neck 2 is substantially cylindrical. However, the neck 2 may comprise a tapered portion or be tapered, as illustrated in FIG. 13. The neck 13 preferably tapers from the atrial side to the aortic side to allow easy insertion as the pump P as it is initially inserted. Once the pump P is inserted the narrower side of neck taper may engage a step on the pump P to prevent the pump P from dislodging. In another embodiment, shown in FIG. 12, the connector 1 may be devoid of a neck and the arms are connected by a connecting means, such as a substantially ring-shaped connecting means. In other words, the neck 2 may simply be a connecting ring.

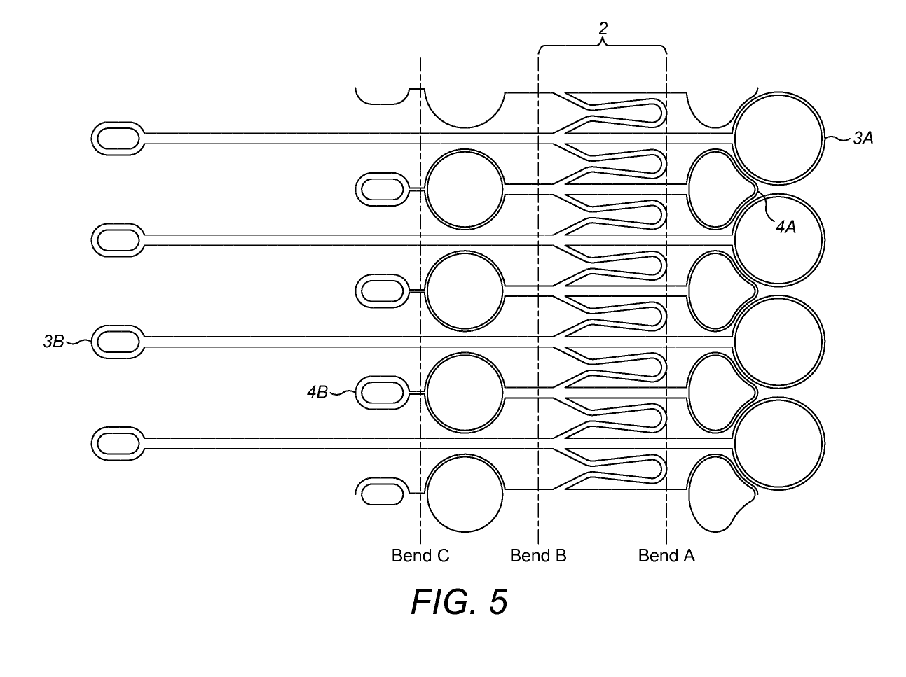

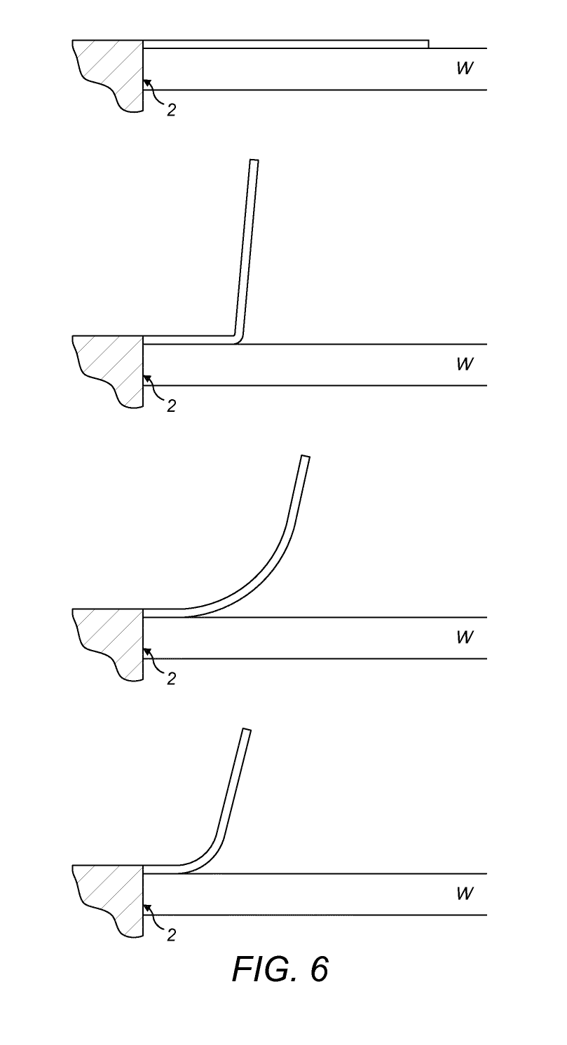

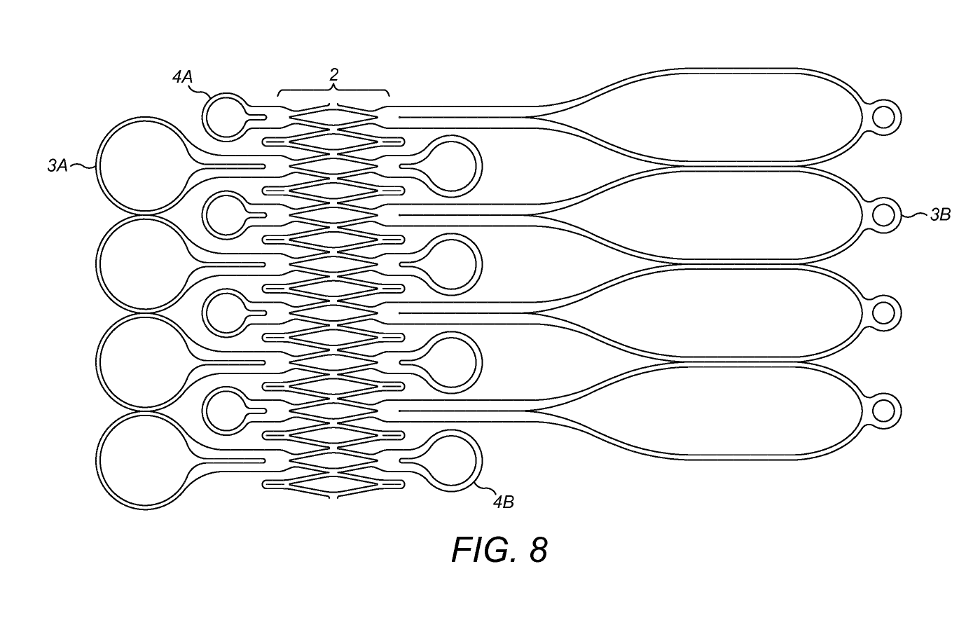

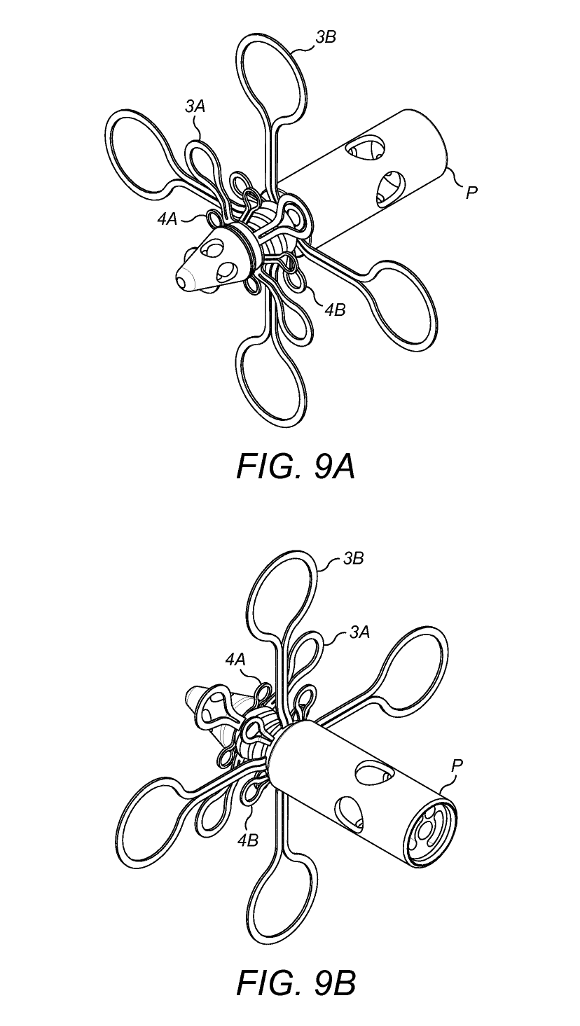

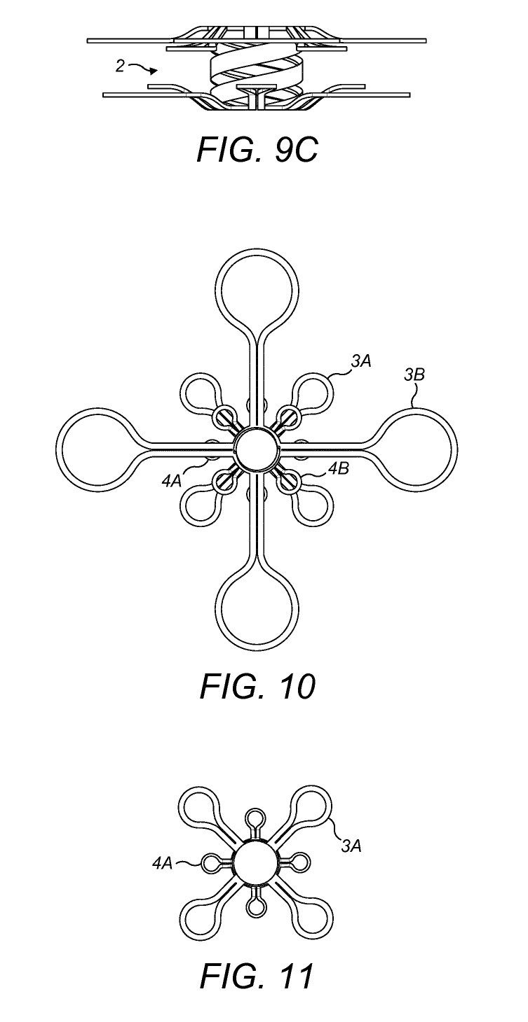

The neck 2 is made of a metal structure for example in the form of woven or shaped wiring. The neck 2 may comprise a repeating pattern which may for example be undulated (including but not limited to serpentine, sinusoidal, triangular, square, rectangular) as shown in FIG. 5 or a diamond-patterned mesh, as shown in FIG. 8. In the embodiment of FIG. 8, the neck 2 comprises a diamond-shaped pattern from which the arms extend longitudinally. Owing to the material and/or the pattern, the neck 2 is capable of expanding radially to accommodate and retain a pump P or other medical device therethrough. The neck 2 is also rigid enough to maintain the tissues surrounding the aperture in the anatomical wall(s) apart and maintaining the neck aperture opened.

With reference to FIGS. 5 and 8, the connector 1 may be cut, for example with a laser, from a sheet or tubing of suitable biocompatible material. The sheet or tubing may be homogeneous in thickness, but, in a preferred embodiment, the sheet or tubing comprises areas of differing thicknesses. For example, an area of greater thickness may be provided to produce the neck section, thereby increasing the radial strength and resistance of the neck 2; an are of thinner thickness may be provided to produce the flexible arms 3.

The outer surface of the neck 2 is covered by an impermeable membrane (not shown) to prevent leakage of blood into the pericardial space, for example in the event that the atrial and aortic walls are not sufficiently pressed against each other. The neck 2 may be coated with a membrane and/or be partially or completely surrounded by an impermeable belt. Suitable materials include, but are not limited to, polymers such as polytetrafluoroethylene (pTFE), silicon, polyvinylidene fluorinated (PVDF) polymers, polyurethane and combinations thereof. Suitable application techniques include, but are not limited to, electro-spinning, eectro-spun, dip coating techniques.

The neck 2 preferably comprises a septum or valve to allow, prevent and/or control the flow of fluid therethrough. The blood flow can therefore be adjusted and controlled, once the connector 1 is suitably implanted and during the delivery and until the implantation of the pump P. The neck 2 may comprise means for retaining the septum or valve, for example the neck 2 may comprise one or more retaining tabs on its inner surface. The neck 2 may comprises means for retaining the pump P and/or any other medical device, for example, the neck 2 may comprise a twist and lock or screw means on its inner surface. It may also be envisaged that the neck 2 comprises a coating membrane (not shown) which forms a valve to seal the opening of the neck 2 prior to the implantation of the intracorporeal pump.

Owing to its shape and structure, the connector according to the present invention is advantageously small or can be compressed to small dimensions such that it is possible to reduce the puncture size. It is easy to manipulate and can be used with patients with compromised or difficult anatomical structures.

A method according to the present invention will now be described by way of example with reference to a left-aorta connection.

The insertion devices (for example guide wire, needle, dilator, sheaths) are inserted by methods known in the art. For example, a needle carrying a guide wire is placed on the groin area of the patient, adjacent the femoral artery. Pressure is applied so that the patient's skin is punctured by the tip of the needle and pushed through the skin and tissues into the femoral artery. Once in place, the guide wire is advanced along the femoral artery and up the inferior vena cava. The guide wire exits the inferior vena cava and enters the right atrium. The septal puncture between the right and left atrium C₁ can also be carried out by methods known in the art. A guide wire now extends from outside the patient, into the femoral artery through the skin puncture, the inferior vena cava, the right atrium, the atrial septum and the left atrium C₁ lodged preferably in superior left pulmonary vein. Next, a large and steerable support sheath 8 can be deployed into the left atrium C₁ over the wire to facilitate the final steps of the procedure. The puncture of the anatomical walls W₁, W₂ is carried out by pushing the atrial wall W₁ against the aortic wall W₂ using the support sheath 8 until the walls are contacting. A needle is pushed against and through the walls to create an opening, which can be subsequently widened using a dilator. A detailed procedure is illustrated in PCT application No. PCT/EP2015/055578.

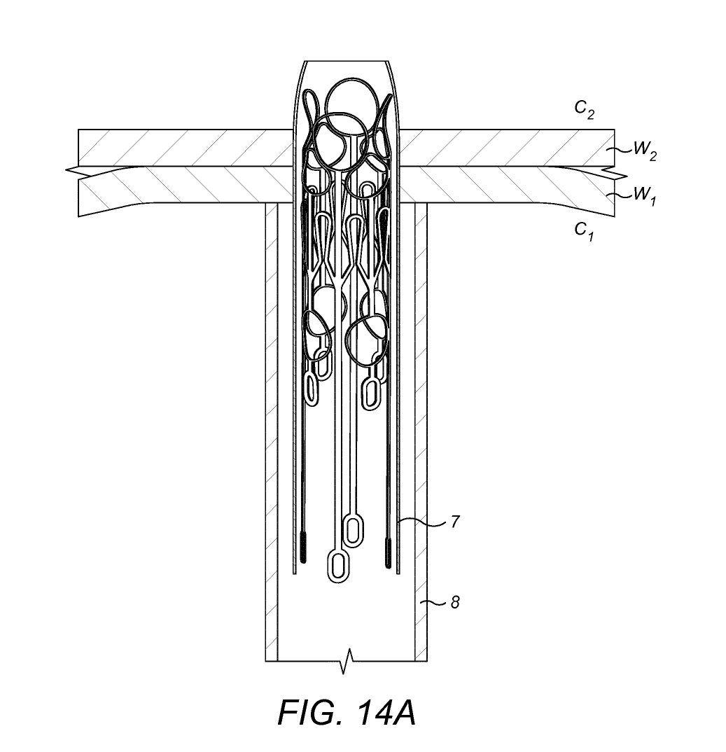

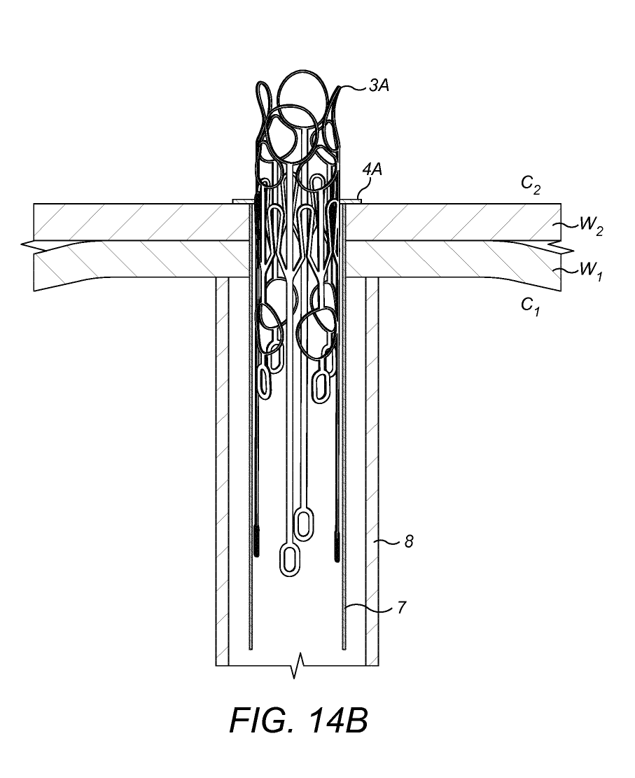

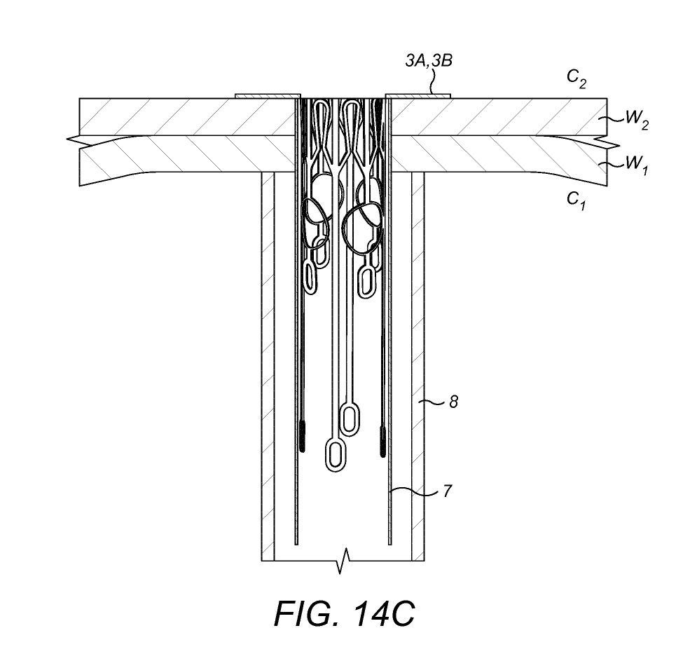

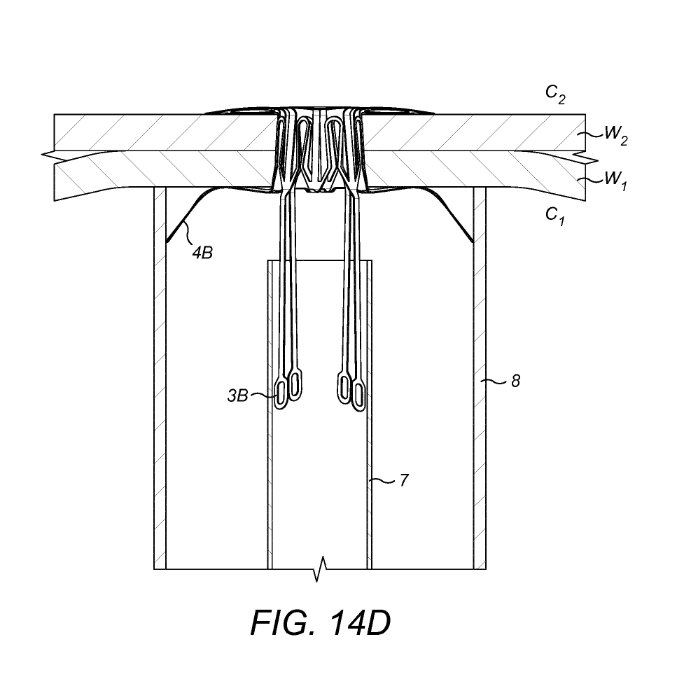

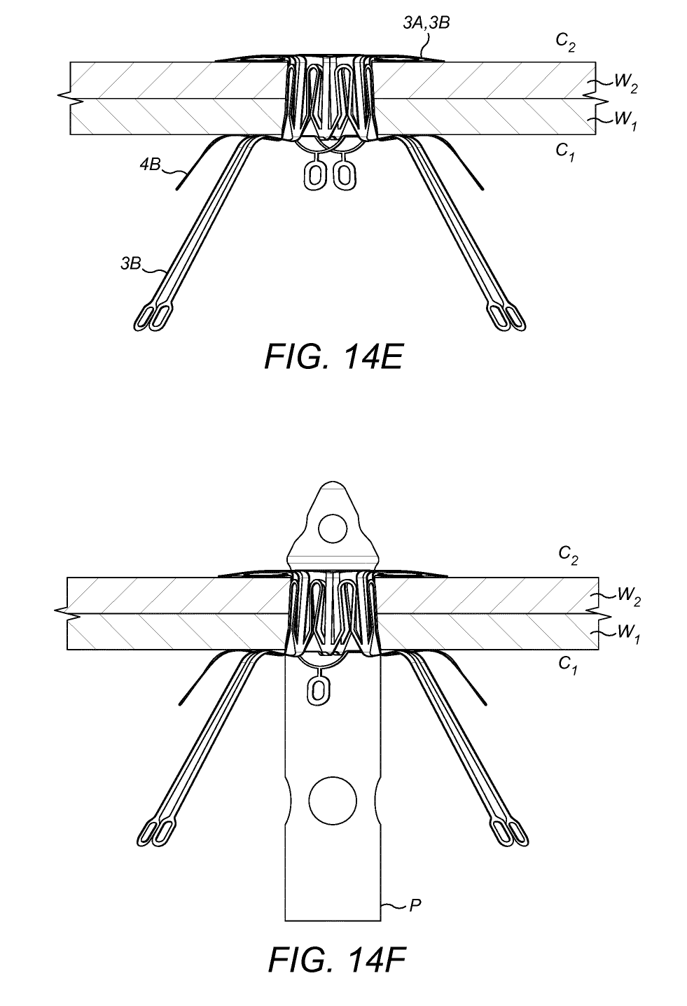

The connector 1 is inserted in the delivery catheter 7 in its delivery configuration. The arms 3A, 3B, 4A, 4B extends substantially parallel from the neck 2 so that they fit within the delivery catheter 7. The catheter 7 is pushed across the anatomical walls W₁, W₂ (FIG. 14A) and the connector 1 is gradually pushed forward (or the catheter is gradually pulled back) so as to exit the catheter 7.

First, the long aortic arms 3A (arms 3A of the primary securing means) partially exit the catheter 7. However, the short aortic arms 4A (arms 4A of the secondary securing means) will be fully exited and deployed first, so as to lie against the aortic wall W₂ and secure the connector to said wall W₂ (FIG. 14B). In this embodiment, the short aortic arms 4A deploy before the long aortic arms 3A, owing to their relatively shorter length. However, other mechanisms can be used to create this sequence. For example, the arms 3A may be shaped and sized so that they are partially or fully deployed in their delivery configuration in the insertion device; the arms 3A may comprise a hinge or other mechanism adapted to deploy arms 3A first.

Secondly, the long aortic arms 3A fully exit the delivery catheter 7 and deploy into their working configuration (FIG. 14C) so as to secure the connector to the aortic wall W₂ and to provide support to said wall W₂. The delivery catheter 7 is gradually pulled back to release the neck 2 of the connector 1, which is now positioned across the anatomical walls W₁ and W₂. Advantageously, the aortic arms 3A and 4A engage the aorta and provide counter traction to pull the aorta A onto the atrium LA during the deployment process.

Once the aortic arms 3A, 4A are deployed, the short atrial arms 4B (arms 4B of the secondary securing means) are released allowing the delivery catheter 7 to remain in contact with the atrial wall W₁ and holding it against the aortic wall W₂. The short atrial arms 4B can now secure the connector 1 to the atrial wall W1 (FIG. 14D) and support the wall W1 until the long atrial arms 3B are deployed.

Finally the long atrial arms (arms 3B of the primary securing means) are deployed, while the small atrial arms maintain pressure on the atrial wall to the aortic wall. The long atrial arms 3B secure the connector 1 to the atrial wall W₁ (FIG. 14E) and to prevent surrounding tissues from hindering the opening of the neck 2. The long aortic arms 3A can prevent soft tissues in the aorta from being sucked into the pump and the long atrial arms 3B can keep the surface expanded to prevent atrial collapse.

Fluid flow can be prevent, allowed and/or controlled by using a septum or valve (not shown) incorporated in the connector 1, for example in the neck 2 of the connector 1. In another embodiment, the neck 2 may comprise or consist of a collapsible membrane, for example an impermeable membrane. In a first step of the implantation process, the connector 1 is secured to the anatomical wall W₂, and as the delivery catheter is pulled back, the neck 2 collapses so that the puncture point is substantially closed and re-opened upon the introduction of a pump P or other medical device. Thus, this feature prevents any fluid passage, until the pump P is inserted.

A pump P or other medical device is delivered to the implantation site through the same or different delivery catheter 7 and coupled to the connector 1 (FIG. 14F). If the connector 1 comprises any retaining means (e.g. twist-and-lock or screw means), then these are used to secure the pump P to the connector 1.

The arms of the connector 1 provide compressive load onto the atrial and aortic wall tissues to seal the connection created and prevent blood leakage external to the heart. The arms 4A, 4B of the secondary securing means can be deployed inside the delivery catheter, for minimum support and to keep both adjacent compartments together until the long arms 3A, 3B are deployed. In addition, a septum or valve of the connector 1 prevents any uncontrolled blood loss, whilst the pump is being delivered, implanted and/or removed.

In a preferred embodiment, the connector 1 according to the present invention is implanted across the anatomical walls using a sheath (support sheath 8 and/or delivery catheter 7) comprising a plurality of distal recesses or slits. The recesses are arranged and configured to facilitate the deployment of the short atrial arms 4B. The long aortic arms 3A and the short aortic arms 4A are deployed as described above; the neck 2 of the connector 1 is positioned across the anatomical walls W₁ and W₂; the distal en of the support sheath 8 with the distal recesses (not shown) pushes the walls W₁ and W₂ against each other. The delivery catheter 7 is slid out of walls W₁ and W₂ to free the short atrial arms 4B. The short atrial arms 4B are deployed and pass through the distal recesses to then lie against anatomical wall W₁. The two walls W₁ and W₂ are now secured and compressed against each between the long aortic arms 3A and the short aortic arms 4A on one side, and the short atrial arms 4B on the other side. The long atrial arms 3B can then be released and deployed. The distal recesses allow the safe implantation of the connector 1 by minimising the risk of blood leakage during the implantation process. This is because the walls W₁ and W₂ can be promptly and accurately secured in a one-step implantation process, without the need for additional stapling or suturing steps prior to connector implantation.

Thus, from the above description, it can be seen that the present invention provides a connector for establishing fluid communication between two anatomical compartments. The connector also enables a pump or other medical devices to be securely implanted across one or more anatomical walls. This can be achieved accurately and safely. The present invention provides a device which can establish fluid communication with minimal risk of blood leakage during the implantation procedure, and whilst providing support to the anatomical walls and tissues so as to prevent injury to the patient.

1-24. (canceled)

25. A method for coupling two anatomical walls using a connector comprising a neck configured to be positioned across the anatomical wall, and primary means for securing the connector across the anatomical walls, and secondary means for securing the connector across the anatomical walls, the method comprising:
securing the neck across the anatomical walls, wherein the connector is secured to the anatomical walls using the secondary securing means prior to securing the connector to the anatomical walls using the primary securing means.

26. The method according to claim 25, wherein the connector can be arranged into a delivery configuration and into a working configuration in which the connector is secured across the anatomical walls.

27. The method according to claim 25, wherein the primary means and/or the secondary means are movable from a delivery position to a working position in which the connector is secured across the anatomical walls.

28. The method according to claim 25, comprising a step of delivering the connector across the anatomical walls using a delivery catheter.

29. The method according to claim 25, wherein the primary securing means comprises a first set of arms and a second set of arms; and the secondary securing means comprises a first set of arms and a second set of arms.

30. The method according to claim 29, comprising a step of deploying the first set of arms of the secondary securing means in the distal compartment.

31. The method according to claim 29, wherein the first set of arms of the secondary securing means is pre-deployed in the delivery catheter.

32. The method according to claim 29, wherein the first set of arms of the secondary securing means have smaller dimensions than the first set of arms of the primary securing means.

33. The method according to claim 29, 32, comprising a step of deploying the first set of arms of the primary securing means in the distal compartment.

34. The method according to claim 29, 33, comprising a step of deploying the second set of arms of the second securing means in the proximal compartment.

35. The method according to claim 29, wherein the second set of arms of the secondary securing means is pre-deployed in the delivery catheter.

36. The method according to claim 29, wherein the first set of arms of the secondary securing means have smaller dimensions than the second set of arms of the primary securing means.

37. The method according to claim 29, comprising a step of deploying the second set of arms of the primary securing means in the proximal compartment.

38. The method according to claim 29, wherein the second set of arms of the primary securing means have smaller dimensions that the second set of arms of the primary securing means.
